(19) United States
(12) Patent Application Publication
(10) Pub. No.: US 2023/0346975 A1
(43) Pub. Date: Nov. 2, 2023

(54) DNA NANOSTRUCTURES FOR BOOSTING IMMUNITY AGAINST CANCER

(71) Applicants: The Johns Hopkins University, Baltimore, MD (US); George Mason University, Fairfax, VA (US)

(72) Inventors: Yun Chen, Baltimore, MD (US); Stavros Gaitanaros, Baltimore, MD (US); Wei-Hung Jung, Baltimore, MD (US); Enze Chen, Baltimore, MD (US); Remi Veneziano, McLean, VA (US)

(21) Appl. No.: 18/247,800

(22) PCT Filed: Oct. 5, 2021
(86) PCT No.: PCT/US2021/053548

§ 371 Date: Apr. 4, 2023

Related U.S. Application Data
(60) Provisional application No. 63/087,931, filed on Oct. 6, 2020.

(51) Int. Cl. A61K 47/69; C12N 15/117; A61K 47/54

(52) U.S. Cl. CPC A61K 47/6957; C12N 15/117; A61K 47/549; A61K 47/6927; A61K 47/6903; C12N 2310/17

(57) ABSTRACT

Described are implantable devices comprising DNA nanostructures comprising a cell ligand at a proximal end tethered to an implantable substrate at a distal end for delivery of CpG oligodeoxynucleotides to cancer cells and methods of use and systems thereof.

### CROSS-REFERENCE TO RELATED APPLICATIONS

This application claims the benefit of U.S. Provisional Application No. 63/087,931, filed Oct. 6, 2020, the content of which is herein incorporated by reference in its entirety.

### TECHNICAL FIELD

The present disclosure relates to a device for delivery of CpG oligodeoxynucleotides to cancer cells and methods of use thereof for the treatment of cancer. In particular, the present disclosure provides an implantable device comprising. DNA nanostructures comprising a cell ligand at a proximal end tethered to an implantable substrate at a distal end.

### BACKGROUND OF THE INVENTION

During cancer development, the innate immune system recognizes damage-associated molecular patterns (DAMP) released from injured tissues to either directly attack the affected cells, or to initiate adaptive immune responses against cancer cells. One of the DAMP is CpG Oligodeoxynucleotides (CpG ODNs), a collection of single-strand DNA (ssDNA) with high content of CpG motifs. The CpG ODNs can trigger TLR9 signaling and subsequent secretion of pro-inflammatory cytokines in innate immune cells, including macrophages, dendritic cells, killer dendritic cells and. natural killer cells. The activated innate immune cells launch attacks on cancer cells, or activate CD4⁺T_{H}1 cells, resulting in cytotoxic CD8⁺T cell to initiate programmed cell death in cancer cells.

Despite the promising therapeutic effect against cancer, adverse effects have been reported, most of which are related to excess immunostimulation causing erythema, pain, swelling, headache, rigors, nausea and vomiting. Long-term treatment using CpG ODNs may lead to autoimmune disorders. The adverse effects caused by systemic stimulation of TLR9 with high doses CpG ODNs can have lethal impact, impairing re-endothelialization upon acute vascular injury and increasing atherosclerotic plaque development. These undesired adverse effects arise from the CpG ODN activated immune cells attacking cancer and normal cells without discrimination, and often compromise the treatment efficacy and quality of life for the patients.

### SUMMARY OF THE INVENTION

The present invention is directed to a device comprising: a plurality of DNA nanostructures tethered to an implantable substrate at a distal end, wherein each of the DNA nanostructures comprises: a cell ligand at a proximal end and CpG oligodeoxynucleotides (CpG ODNs) configured to be released when the DNA nanostructure is under force.

The present invention is also directed to a method for treating cancer comprising implanting a device as disclosed herein in a subject in need thereof.

Other aspects of the invention will become apparent by consideration of the detailed description and accompanying drawings.

### BRIEF DESCRIPTION OF THE DRAWINGS

The patent or application file contains at least one drawing executed in color. Copies of this patent or patent application publication with color drawings will be provided by the Office upon request and payment of the necessary fee.

FIGS. 1A-1B show DNA nanobeam constructs with prescribed nick and HJs densities to be subjected to stretching using hydrodynamic forces. FIG. 1A is schematics of DX-based designs of two-helix beams. In construct C85N a double crossover was placed every 42 nucleotides (85 HJs total) while in C170N the crossover occurred every 21 nucleotides (170 HJs total). FIG. 1B is a TEM image showing that the length of the nanobeams was approximately 1.2 μm, as expected from the design. Scale bar: 100 nm.

FIGS. 2A-2D show flow-induced stretching of DNA origami and particle tracing set-up. FIG. 2A is a schematic of the experimental design of the axial stretching of the DNA nanobeams using microfluidics. One end of the nanobeam was conjugated with digoxigenin and then immobilized on an anti-digoxigenin antibody-grafted glass surface. The free end of the DNA nanobeam was conjugated with biotin and then bound to a streptavidin-coated, micron-sized particle. The stretching force was applied hydrodynamically through the particle by flowing the buffer through the microfluidic device at specified rates. FIG. 2B is representative time-lapse images showing the displacement of the micron-sized particle bound to the DNA nanobeam by the flow. The XY coordinate of the centroid (red asterisk) was recorded as the DNA nanobeam stretched along the direction of the flow (arrow) at various rates, imposing different magnitudes of stretching forces. Timestamp format is mm:ss. Scale bar: 1 μm. FIG. 2C is a graph of flow rates ranging from 100-1300 μL/min, generated by a syringe pump exerting approximately 5-65 pN as the particle drag force, used to stretch the DNA nanobeams. FIG. 2D is a graph of the representative displacement plotted against time for the C170L nanobeam-bound particle.

FIGS. 3A-3B show that the presence of nicks and HJs reduced the axial stiffness of DNA nanobeams. FIG. 3A are graphs of representative force-displacement responses of constructs C85L, C85N, C170L and C170N. Four combinations of nanobeam pairs are plotted to illustrate how nicks and HJs reduce the apparent stiffness. FIG. 3B is a graph of axial stiffness (k) values of C85L, C85N, C170L and C170N. Construct CSSL, the ligated nanobeam with the least HJs was the stiffest among all four constructs.

FIGS. 4A-4D show continuum modeling predicted force-displacement responses agreeing with experimental measurements and showed how the stiffness of multi-helix bundles compares to the one of the double helix. FIG. 4A is a predicted model plotted against the measurements corresponding to constructs C85L; C85N; C170L, C170N, and the 6-helix and 10-helix nanobeams as previously reported (Pfitzner, E., et al. (2013) Angew. Chemie, 125, 7920-7925, incorporated herein by reference in its entirety). FIG. 4B is a graph of local stiffness values at the locations of and nicks are compared to the intact double helix. Optimized fitting showed a corresponding local stiffness for HJs and nicks being 0.023- and 0,0081-fold respectively of the value of intact B-form DNA. FIG. 4C is a graph of the force-displacement curves from tensile simulations on origami beams with cross-sections consisting of two; six, nine, ten, and sixteen helices, representing by blue, orange, yellow, green, and gray lines, respectively. FIG. 4D is a graph of the stiffness values for the five origami beams simulated in FIG. 4C, normalized to the stiffness of B-form DNA, and shown as a function of the number of helices in each packing.

FIG. 5 is a schematic of the principle of one embodiment of the disclosed device. The DNA nanostructure will release CpG ODNs only when cancer cells exert strong forces to deconstruct or disassemble the DNA nanostructure.

FIG. 6 is a schematic showing the selectivity of the delivery to cancer cells over normal cells. The strong forces created during cell migration of cancer cells disassembles or deconstructs the DNA nanostructures, releasing CpG ODNs which are then bound by the cancer cells thereby tagging the cancer cells for attack by immune cells.

### DETAILED DESCRIPTION OF THE INVENTION

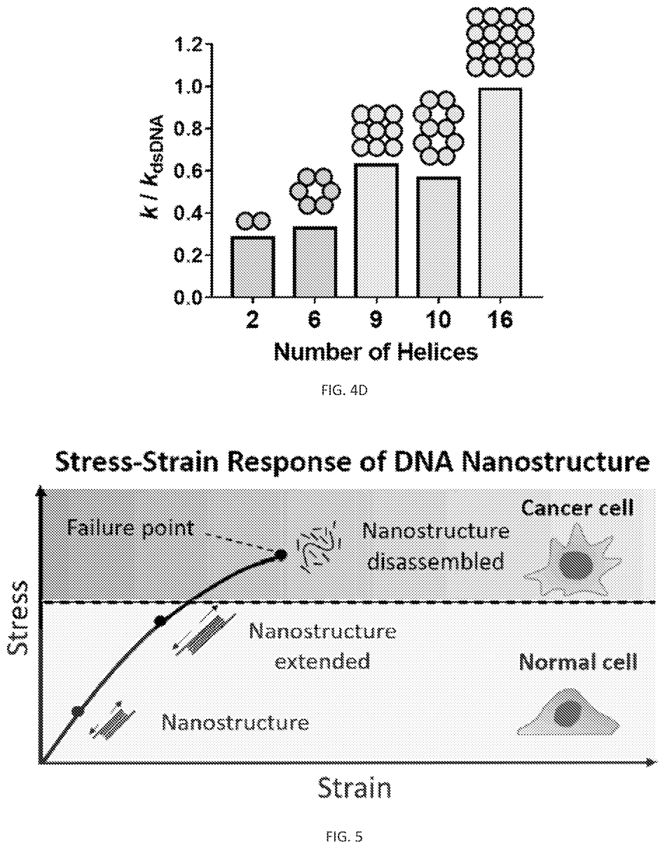

Disclosed herein is a device containing artificially synthesized DNA nanostructures, including but not limited to DNA origami, that release CpG ODNs only when a strong force is exerted to deconstruct the DNA nanostructures. In particular, the device has an implantable substrate containing DNA nanostructures immobilized on its surface. The DNA nanostructures may be conjugated with a ligand which binds to a specific biomarker expressed abundantly on targeted cells (e.g., cancer cells). The CpG ODNs will only be released by cancer cells generating high forces causing disassembly of the DNA nanostructure (FIG. 5). Thus, the device facilitates two different methods of ensuring specificity: first, the ligand binds to a specific biomarker present more abundantly on the surface of the targeted cells than non-targeted cells, and second, cancer cells generate the force needed to disassemble the DNA nanostructures to release CpG ODNs while normal cells would not generally create the level of force capable of disassembling the DNA nanostructures.

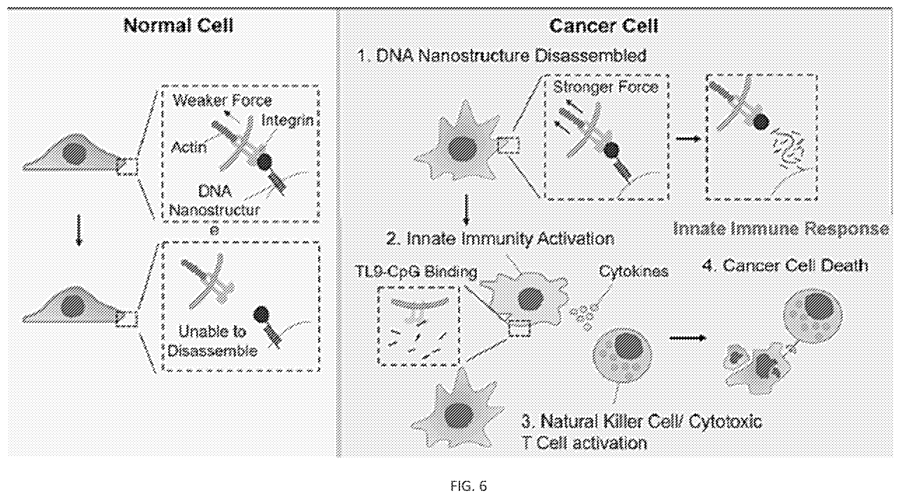

Cancer cells bind the CpG ODN resulting in phagocytosis by both macrophages and dendritic cells (DCs), resulting in the activation of CD4⁺T_{H}1 cells. In contrast, normal cells also expressing the biomarker, though to a lesser degree, will not activate the immune system and be attacked subsequently (FIG. 6). The device may be implanted in tissue during tumor removal surgery. The device may be used in conjunction with other cancer treatments, including immunotherapy, radiation and chemotherapy. The disclosed device improves the specificity and reduces the adverse systemic effects of CpG ODN treatment seen in the conventional CpG ODN-based immunotherapy.

### 1. Definitions

Unless otherwise defined, all technical and scientific terms used herein have the same meaning as commonly understood by one of ordinary skill in the art. In case of conflict, the present document, including definitions, will control. Preferred methods and materials are described below, although methods and materials similar or equivalent to those described herein can be used in practice or testing of the present invention. All publications, patent applications, patents and other references mentioned herein are incorporated by reference in their entirety. The materials, methods, and examples disclosed herein are illustrative only and not intended to be limiting.

The terms “comprise(s),” “include(s),” “having,” “has,” “can,” “contain(s),” and variants thereof, as used herein, are intended to be open-ended transitional phrases, terms, or words that do not preclude the possibility of additional acts or structures. The singular forms “a,” “an” and “the” include plural references unless the context clearly dictates otherwise. The present disclosure also contemplates other embodiments “comprising,” “consisting of,” and “consisting essentially of” the embodiments or elements presented herein, whether explicitly set forth or not.

The modifier “about” used in connection with a quantity is inclusive of the stated value and has the meaning dictated by the context (for example, it includes at least the degree of error associated with the measurement of the particular quantity). The modifier “about” should also be considered as disclosing the range defined by the absolute values of the two endpoints. For example, the expression “from about 2 to about 4” also discloses the range “from 2 to 4.” The term “about” may refer to plus or minus 10% of the indicated number. For example, “about 10%” may indicate a range of 9% to 11%, and “about 1” may mean from 0.9-1.1. Other meanings of “about” may be apparent from the context, such as rounding off, so, for example “about 1” may also mean from 0.5 to 1.4.

For the recitation of numeric ranges herein, each intervening number there between with the same degree of precision is explicitly contemplated. For example, for the range of 6-9, the numbers 7 and 8 are contemplated in addition to 6 and 9, and for the range 6.0-7.0, the number 6.0, 6.1, 6.2, 6.3, 6.4, 6.5, 6.6, 6.7, 6.8, 6.9, and 7.0 are explicitly contemplated.

“Cell,” as used herein, refers to the basic functional unit of life, and includes both prokaryotic and eukaryotic cells. Cells are characterized by an interior having the nucleus or nucleoid, and a cell membrane (cell surface).

As used herein, the term “chemotherapeutic” or “anti-cancer drug” includes any drug used in cancer treatment or any radiation sensitizing agent. Chemotherapeutics may include alkylating agents (including, but not limited to, cyclophosphamide, mechlorethamine, chlorambucil, melphalan, dacarbazine, nitrosoureas, and temozolomide), anthracyclines (including, but not limited to, daunorubicin, doxorubicin, epirubicin, idarubicin, mitoxantrone, and valrubicin), cytoskeletal disrupters or taxanes (including, but not limited to, paclitaxel, docetaxel, abraxane, and taxotere), epothilones, histone deacetylase inhibitors (including, but not limited to, vorinostat and romidepsin), topoisomerase inhibitors (including, but not limited to, irinotecan, topotecan, etoposide, teniposide, and tatiuposide), kinase inhibitors (including, but not limited to, bortezomib, erlotinib, gefitinib, imatinib, vemurafenib, and vismodegib), nucleotide analogs and precursor analogs (including, but not limited to, azacitidine, azathioprine, capecitabine, cytarabine, doxifluridine, fluorouracil, gemcitabine, hydroxyurea, mercaptopurine, methotrexate, and tioguanine), peptide antibiotics (including, but not limited to, bleomycin and actinomycin), platinum-based agents (including, but not limited to, carboplatin, cisplatin and oxaliplatin), retinoids (including, but not limited to, tretinoin, alitretinoin, and bexarotene), vinca alkaloids and derivatives (including, but not limited to, vinblastine, vincristine, vindesine, and vinorelbine), or combinations thereof. The chemotherapeutic may in any form necessary for efficacious administration and functionality. “Chemotherapy” designates a therapeutic regimen which includes administration of a “chemotherapeutic” or “anti-cancer drug.”

“Complementarity” or “complementary to,” as used herein, refer to the ability of a nucleic acid to form hydrogen bond(s) with another nucleic acid sequence by either traditional Watson-Crick or other non-traditional types.

As used herein, “DNA nanotube” refers to a structure composed of individual units that form from five short DNA strands that self-assemble to form a rigid, brick-like structure, called a monomer because of a preference for Watson-Crick complementarity. Each of these basic units, or monomers has four locations where they join to similar sites on other monomers in a pattern that allows them to assemble to form a repeating structure, or polymer. The specificity of DNA hybridization and the predictable sequence-independent structure of the DNA double helix has enabled assembly of DNA nanotubes while controlling structure, circumference, and length, as well as functionalization to connections to a variety of other materials. See, for example, Rothemund, P. W. K., et al., J. Am. Chem. Soc. 2004, 126, 16344-16352, Liu D. et al, Proc. Natl. Acad. Sci. U.S.A., 2004 101 (3) 717-722, Aldaye, F. A., et al., Nat. Nanotechnol. 2009, 4, 349-352, Mitchell, J. C., et al., J. Am. Chem. Soc. 2004, 126. 16342-16343, Wilner, O. I., et al., Nat. Commun. 2011, 2, 540., Liu H. et al, Angew Chem Int Ed Engl. 2006 Mar 13;45(12):1942-5, Yin, P., et al., Science 2008, 321, 824-826, Douglas, S. M., et al., Proc. Natl. Acad. Sci. U.S.A. 2007, 104, 6644-6648, Aldaye, F. A., et al., Science 2008, 321, 1795-1799, Bui, H., et al., Nano Lett. 2010, 10, 3367-3372, Sharma, J. et al., Science 2009, 323, 112-116, Shen, X., et al., J. Am. Chem. Soc. 2011, 134, 146-149, Mohammed, A. M. and Schulman, R. Nano Letters 13 (9) 4006-4013, 2013, Mohammed, A. M., et al., Nature Nanotechnology 12, 312-316, 2017; and Mohammed, A. M., et al., Nanoscale, 9, 522-526, 2017, all of which are incorporated herein by reference. Any of these methods, as well as those described elsewhere in the specification, may be used to form the DNA nanotubes used herein. In some embodiments, nanotubes can be assembled using the monomers formed by RNA strands or RNA/DNA strands. These monomers will assemble RNA nanotubes or RNA/DNA hybrid nanotubes , see, for example, Stewart J. M. et al, ACS Nano 2019, 13, 5, 5214-5221, Agarwal S. et al, J. Am. Chem. Soc. 2019, 141, 19, 7831-7841.

“Hybridization” refers to a reaction in which one or more polynucleotides react to form a complex that is stabilized via hydrogen bonding between the bases of the nucleotide residues. The hydrogen bonding may occur by Watson Crick base pairing, Hoogstein binding, or in any other sequence specific manner according to base complementarity. The complex may comprise two strands forming a duplex structure, three or more strands forming a multi stranded complex, a single self-hybridizing strand, or any combination of these. A second sequence that is complementary to a first sequence is referred to as the “complement” of the second sequence. The term “hybridizable” as applied to a polynucleotide refers to the ability of the polynucleotide to form a complex that is stabilized via hydrogen bonding between the bases of the nucleotide residues in a hybridization reaction.

The term “microparticle” as used herein refers to any particulate carrier, usually greater than 1 micron in size, which is used for the delivery of biologically active materials, e.g., the DNA nanostructures described herein. The microparticle may include microcapsules, microspheres, and the like, whether natural or artificial.

“Polynucleotide” or “oligonucleotide” or “nucleic acid,” as used herein, means at least two nucleotides covalently linked together. The polynucleotide may be DNA, both genomic and cDNA, RNA, or a hybrid, where the polynucleotide may contain combinations of deoxyribo- and ribo-nucleotides, and combinations of bases including uracil, adenine, thymine, cytosine, guanine, inosine, xanthine hypoxanthine, isocytosine and isoguanine. Nucleic acids may be obtained by chemical synthesis methods or by recombinant methods. Polynucleotides may be single- or double-stranded or may contain portions of both double stranded and single stranded sequence. The depiction of a single strand also defines the sequence of the complementary strand. Thus, a nucleic acid also encompasses the complementary strand of a depicted single strand. Many variants of a nucleic acid may be used for the same purpose as a given nucleic acid. Thus, a nucleic acid also encompasses substantially identical nucleic acids and complements thereof.

A “peptide” or “polypeptide” is a linked sequence of two or more amino acids linked by peptide bonds. The peptide or polypeptide can be natural, synthetic, or a modification or combination of natural and synthetic. Polypeptides include proteins such as binding proteins, receptors, and antibodies. The proteins may be modified by the addition of sugars, lipids or other moieties not included in the amino acid chain. The terms “polypeptide” and “protein,” are used interchangeably herein. Any compositions or methods provided herein can be combined with one or more of any of the other compositions and methods provided herein.

Preferred embodiments of this invention are described herein, including the best mode known to the inventors for carrying out the invention. Variations of those preferred embodiments may become apparent to those of ordinary skill in the art upon reading the foregoing description. The inventors expect skilled artisans to employ such variations as appropriate, and the inventors intend for the invention to be practiced otherwise than as specifically described herein. Accordingly, this invention includes all modifications and equivalents of the subject matter recited in the claims appended hereto as permitted by applicable law. Moreover, any combination of the above-described elements in all possible variations thereof is encompassed by the invention unless otherwise indicated herein or otherwise clearly contradicted by context.

2. Implantable Device

The present disclosure provides a device comprising a plurality of DNA nanostructures linked to an implantable substrate at a distal end, The DNA nanostructures comprise CpG oligodeoxynucleotides (CpG ODNs) configured to be released when the nanostructure is under force. The DNA nanostructures comprise a ligand at a proximal end (for example, a cancer cell ligand). The device is implantable or insertable into a subject.
a) DNA Nanostructures

DNA nanostructures are materials constructed by leveraging self-assembly of single-stranded DNA (ssDNA) strands having sequences that have predetermined Watson-Crick base pairing complementarity to other ssDNA strands such that the collective hybridization of these strands may produce a wide variety of well-ordered DNA nanoarchitectures with well-defined shapes and sizes. The DNA nanostructures may comprise scaffolded DNA structures, also known as DNA origami, or tile- or brick-based DNA nanostructures, Scaffolded DNA structures comprise one long ssDNA molecule called the scaffold, which is typically the ˜7 kilobase pair (kbp) ssDNA genome of the m13 bacteriophage, which is folded into a desired shape using short (24-60 bp) ssDNA molecules called staple strands. Tile- or brick-based DNA nanostructures lack one single unifying scaffold strand and rather are assembled from a set of short ssDNA strands. The DNA nanostructures may include by are not limited to, helix bundles, DNA nanotubes, DNA nanobeams, and the like. See, for example, Kauert, D. J., Kurth, T., Liedl, T. and Seidel, R., 2011, Nano letters, 11(12), pp.5558-5563, Sun, W., Boulais, E., Hakobyan, Y., Wang, W. L., Guan, A., Bathe, M. and Yin, P., 2014. Science, 346(6210), and Pfitzner, E., Wachauf, C., Kilchherr, F., Peitz, B., Shih, W. M., Rid, M. and Dietz, H., 2013. Angewandte Chemie International Edition, 52(30), pp.7766-7771, each incorporated herein by reference in their entirety. In some embodiments, DNA nanostructures can be assembled using the monomers formed by RNA strands or RNA/DNA strands.

In some embodiments, the DNA nanostructures comprise DNA nanobeams. DNA nanobeams are helix bundles consisting of at least 2 helices, which connect to one another through Holiday junctions. A single helix is formed by a plurality single-strand DNAs of varied length and intertwined with other helix bundles at the Holliday junctions.

In some embodiments, the CpG ODNs are staple strands within an individual DNA nanostructure. The DNA nanostructure may comprise staple strands that are not CpG ODNs. In some embodiments, the CpG ODNs are other ssDNA strands within an individual DNA nanostructure.

Immobile Holliday junctions (HJs) are crossovers that connect neighboring DNA helices are the main motif of all DNA nanostructures (2), Two HJs can be combined to create a double crossover motif (DX-tile)(18), which significantly increases the rigidity of DNA nanostructures and has thus been the key building block in many self-assembled scaffolds, both origami- and tile-based ones (19-22). The distance between consecutive HJs in the DX-tile is typically chosen to be 2nZ nucleotides, where n is the period of B-form DNA (10.5 nucleotides) and Z is an integer. This choice leads to minimal distortions that can arise from the development of internal bending and torsional moments. The axial stiffness of DNA nanostructures (e.g., DNA nanobeams) can be varied by changing the density of HJs and nicks, such that the force needed to deconstruct the DNA nanostructure is able to be varied. The DNA nanostructures are configured to deconstruct and release CpG ODNs when under force, such as the higher forces between a cancer cell and its ligand that occur during cell migration. The exact number of HJs and nicks, and thereby the axial stiffness value, may be adjusted based for different subtypes of the diseases and/or individual patients. In general, higher densities of HJs and nicks decreases axial stiffness. In addition, the ultimate tensile strength (UTS) or the stress required to unfold the DNA nanostructure can also be adjusted by varying the nicks and HJs throughout the DNA nanostructure.

The DNA nanostructures may comprise a cell ligand at a proximal end. The cell ligand may comprise any molecule known to bind a cell surface protein or receptor on the targeted cell (e.g., a cancer cell), thus linking the DNA nanostructure, and, by extension, the device, to the cell. The cell ligand may be a polypeptide, glycoprotein, nucleic acid, small molecule, carbohydrate, lipid, etc. For example, a cell ligand can be a nucleic acid (e.g. an aptamer, Spiegelmer®, etc.) that binds to a cell type specific marker. In general, an aptamer is an oligonucleotide (e.g., DNA, RN-A, or an analog, derivative, or combination thereof) that binds to a particular target, such as a polypeptide. The cell ligand may be a naturally occurring or synthetic ligand for a cell surface receptor, e.g., a growth factor, hormone, LDL, transferrin, etc. The cell ligand may be an antibody or any characteristic fragment thereof. Synthetic binding proteins such as Affibodies®, Nanobodies™, AdNectins™, Avimers™, etc., may be used. Peptide and non-antibody protein cell ligands can be identified, e.g., using procedures such as phage display (e.g., RGD peptides, NGR peptide, and transferrin LHRH). This widely used technique has been used to identify cell specific ligands for a variety of different cell types. The small molecules may include synthetic or natural molecules which target specific receptors or binding partners (e.g., folate, galactose)

In some embodiments, the ligand is a cancer cell ligand. A cancer cell ligand is a ligand which preferentially binds to cancer cells over non-diseased or normal cells. The cancer cell ligand may be specific for a certain type of cancer (e.g., breast cancer, colorectal cancer, lung cancer). For example, cancer cell ligands may bind to: receptor tyrosine-protein kinase erbB-2, also known as CD340 (cluster of differentiation 340), proto-oncogene Neu, Erbb2 (rodent), or ERBB2 (human); epidermal growth factor receptor (EGFR, ErbB-1; HER1 in humans) or B-lymphocyte antigen CD20 or CD20.

In some embodiments, the cell ligand comprises fibronectin. In some embodiments, the cell ligand comprises a tri-amino acid, arginine-glycine-aspartate (RGD) peptide. In some embodiments, the cell ligand comprises cluster of differentiation 80 protein (CD80).

In some embodiments, the cancer ligand may bind to cytotoxic T-lymphocyte-associated protein 4 (CTLA-4). In some embodiments, the cell ligand may bind to integrin.

In some embodiments, the DNA nanostructures comprise a functional group to which the cell ligand may be conjugated. Functional group pairs are well-known in the art and suitable for use with the device described herein.
b) Implantable Substrate

The device described herein comprises DNA nanostructures tethered to an implantable substrate at a distal end. In some embodiments, the DNA nanostructures are tethered to the implantable substrate with a covalent bond. In some embodiments, the DNA nanostructures are tethered to the implantable substrate with a noncovalent bond.

The implantable substrate may comprise an agent on an outer surface that reaction or binding to a moiety on the distal end of the DNA nanostructure.

In some embodiments, the agent and the moiety are a binding pair. A binding pair refers to a pair of molecules comprising a binding member and a binding partner which have particular specificity for each other and under normal conditions bind to each other in preference to binding to other molecules. The interaction of the binding pair is typically non-covalent. The binding member and binding partner may comprise a part of a larger molecule.

The binding pair may include protein:protein binding pairs (e.g., protein:antibody, such as biotin:avidin,), protein:polynucleotide binding pairs, protein:carbohydrate binding pairs, protein:small molecule binding pairs, polynucleotide:polynucleotide binding pairs, and the like. Examples of a specific binding pair include an antibody and an antigen, biotin and avidin or streptavidin, a ligand and a receptor, a lectin and a carbohydrate, an enzyme and a cofactor or substrate, oppositely charged ionic groups, redox/electrochemical groups, a chelating group and its binding partner, or a nucleic acid molecule capable of hybridising to a complementary nucleic acid sequence.

In some embodiments, the agent and the moiety are functional groups that react to forth a covalent bond. For example, functional groups that facilitate bioconjugation reactions (e.g., thiol conjugation reactions, amine-modified DNAs with carbonyl functional groups, and the like). For exemplary agent:moiety pairs see Kalia J, Raines R T. Curr Org Chem. 2010;14(2):138-147, Mukesh Digambar Sonawane, Satish Balasaheb Nimse, Journal of Chemistry, vol. 2016, Article :ID 9241378, 19 pages, 2016, and Bioconjugate Techniques, Ed. Hermanson, G T, Academic Press, 1996, Pages 727-728, ISBN 97801234233:51, each incorporated herein by reference in its entirety.

Any implantable substrate suitable for insertion into a subject's body, whether on a temporary or a permanent basis, may be used with the disclosed device. The implantable substrates described herein may be made from any biocompatible material suitable for implantation. The implantable substrate may include, but is not limited to: soft tissue repair devices such as sutures, staples, meshes, patches, clips, clamps, screws, and pins; staple line reinforcements; tissue fillers; tissue wraps for solid organs or luminal structures; sealing devices; cavity wall and floor reinforcements, intramuscular conduits; access site closure devices; prosthetics; stents; shunts; catheters and catheter-like devices; hydrogels; porous silicone gel implants; and the like. In some embodiments, the implantable substrate comprises a catheter-like device, a shunt (e.g. a bypass shunt), or a porous silicone gel. In some embodiments; the DNA nanostructure is conjugated with a micron-sized particle or microparticle. The microparticle may be coated with targeting moieties recognizable by target cells or target tissues, e.g., diseased (e.g., cancer) cells, The targeting compound facilitates engagement between the diseased cell and the DNA nanostructure. The targeting compound may be any ligand or binding agent preferentially able to bind the desired target cells or target tissues. The targeting compound may be a polypeptide, glycoprotein, nucleic acid, small molecule, carbohydrate, lipid, etc.

### 3. Method of Treating Cancer

The present disclosure provides methods of treating cancer in a subject comprising implanting the device as described herein in a subject in need thereof. Descriptions of the inventive device set forth above are also applicable to the method of treating cancer.

The methods can be used with any cancer cell or in a subject having any type of cancer, for example those described by the National Cancer institute. The cancer may be a carcinoma, sarcoma, lymphoma, leukemia, melanoma, mesothelioma, multiple myeloma, or seminoma. The cancer may be a cancer of the bladder, blood, bone, brain, breast, cervix, colon/rectum, endometrium, head and neck, kidney, liver, lung, muscle tissue, ovary, pancreas, prostate, skin, spleen, stomach, testicle, thyroid or uterus.

In some embodiments, the cancer is a solid tumor. Examples of cancers that are solid tumors include, but are not limited to, brain, pancreatic, bladder, colon, non-small cell lung cancer (NSCLC), breast and ovarian cancers.

The device may be implanted in a tumor or lesion, tissue proximate or surrounding the tumor, or a cavity region created by tumor excision or resection (such as the surrounding tissue or cavity associated with tumor resection/excision). For example, the device may be implanted into and/or around a tumor before surgical excision or after removing tumor tissue to treat the surrounding tissue post-operatively.

A wide range of second therapies may be used in conjunction with the device of the present disclosure. The second therapy may be a combination of a second therapeutic agent or may be a second therapy not connected to administration of another agent. Such second therapies include, but are not limited to, immunotherapy, radiotherapy, or administration of a second chemotherapeutic agent.

### 4. System

The present disclosure provides a system comprising at least one or all of an implantable substrate, a plurality of DNA nanostructures comprising CpG oligodeoxynucleotides (CpG ODNs) configured to be released when the DNA nanostructure is under force, and a cell ligand. Each DNA nanostructure is configured to be tethered to the implantable substrate at a distal end and the cell ligand at a proximal end. Descriptions of the implantable substrate, the DNA nanostructures and the cell ligand set forth above are also applicable to the disclosed system.

Individual member components of the system may be physically packaged together or separately. The components of the system may be provided in bulk packages (e.g., multi-use packages) or single-use packages. The systems can also comprise instructions for using the components of the system. The instructions are relevant materials or methodologies pertaining to the system. The materials may include any combination of the following: background information, list of components and their availability information (purchase information, etc.), brief or detailed protocols for using the compositions, trouble-shooting, references, technical support, and any other related documents. Instructions can be supplied with the system or as a separate member component, either as a paper form or an electronic form which may be supplied on computer readable memory device or downloaded from an internee website, or as recorded presentation. It is understood that the disclosed system can be employed in connection with the disclosed methods. The systems or components therein may be provided in suitable packaging.

### 5. Examples

Materials and Methods

DNA origami assembly DNA staple strands were purchased from IDT and used directly for folding (Jung et al. Nucleic Acids Research, 2020, Vol. 48, No. 21, 12407-12414, incorporated herein by reference in its entirety). The M13 Bacteriophage single-stranded DNA was used as scaffold for origami construction. In a typical sample preparation, staple strands were mixed with the ssDNA scaffold (p7490 from Guild Biosciences) at 10 nM final concentration in 10-fold molar excess (100 nM) in folding buffer (5 mM Tris base, 1 mM EDTA, supplement with 14 mM MgCl₂) in a total volume of 50 μL. The solution was slowly annealed from 95° C. down to room temperature (24° C.) in a PCR thermal cycler overnight using the following program: 95° C. for 5 minutes, 80° to 70° C. at 1° C. per 5 minutes, 70° to 30° C. at per 15 minutes, and 30° to 25° C. at 1° C. per 10 minutes. For the purpose of conjugating the DNA nanobeams to the surface of the glass chamber via anti-digoxigenin antibody and to the gold nanoparticles coated with streptavidin, the staple strands positioned at both ends of the DNA nanobeams were conjugated with either biotin or digoxigenin.

Ligation of consecutive DNA staple strands The protocol used for ligation of the DNA origami constructs was adapted from Wang et al. (Wang, J. and Lu, C. (2007) J. Appl. Phys., 102, 074703, incorporated here by reference in its entirety). Briefly, after annealing and purification of the nanostructures with the Biorad PCR Kleen purification system to remove the excess of staple strands, the nanostructures were incubated with the T4 Polynucleotide Kinase to ensure phosphorylation of all 5′-ends. The incubation was performed at 37° C. for 4 hours in the provided T4 ligase buffer. After the 4 hours of incubation, T4 DNA ligase was added to the mix to ligate ail nicks of the structures and the sample was incubated at 37° C. for an extra hour.

Gel electrophoresis Folded DNA origami constructs were subjected to 1.5% native agarose gel electrophoresis and run at 70V for 2 hours (gel prepared in 0.5×TBE buffer supplemented with 11 mM MgCl2 and 0.005% (v/v) EtBr) in an ice water bath. The image was acquired with an Azure C150 gel documentation system (Azure Biosystems). Following the gel electrophoresis, the target gel bands were excised and placed into a Freeze 'N Squeeze column (Bio-Rad Laboratories, Inc.). The excised gel was crushed into fine pieces by a microtube pestle in the column, and the column was then centrifuged at 7000 g for 5 minutes.

TEM preparation The DNA origami construct (C170L) with concentration of 10 ng/μL was used for TEM imaging. Two microliters of the sample were adsorbed for 2 minutes onto plasma treated carbon-coated TEM grids. The grids were then stained for 10 seconds using a 1% aqueous uranyl acetate solution and followed by 3 times 10 seconds washing using deionized water and air drying. Imaging was performed using a FEI F200C Talos 200keV FEG transmission electron microscope,

Flow Chamber Preparation Flow chambers were made by bonding air-plasma treated polydimethylsiloxane (PDMS) (Dow Coming, Sylgard 184) slabs to 22×50 mm cover glass (Corning, 2975-225); the PDMS slabs were cast over scotch tape strips (3M, Scotch® Magic™ tape 810) to form shallow channels with dimension of 15 mm×1 mm×0.06 mm (length×width×height, respectively). Inflow and outflow ports were formed by puncturing the PDMS with harris punch (Harris Uni-Core I.D. 4 mm). To immobilize DNA origami, flow chambers were filled with 0.1 μM polyclonal sheep anti-digoxigenin antibody (Sigma-Aldrich, 11333089001) in phosphate buffered saline (PBS) (ThermoFisher Scientific, 10010049) and incubated for 2 hours to non-specifically adsorb anti-digoxigenin antibody to the cover glass surfaces, After washing by flowing PBS for 30 minutes, the blocking solution with 3 mg/mL bovine serum albumin (Sigma-Aldrich, A9647) and 0.1% Tween 20 (Promega, H5152) in PBS was then flowed into the flow chambers and incubate for additional two hours. The blocking solution was then removed and the DNA beam solution with a concentration of 100 ng/μL was then added to the flow chambers for overnight at 4° C. The flow chambers were washed with PBS 2 hours the next day. Finally, 20 μL of the 1 μm streptavidin coated fluorescent particle (dragon green) (Bangs Laboratories, CFDG004) with density of 4.7×10⁹ particles/mL was added to each channel and incubated for 2 hours. These flow chambers were then used for the following experiments.

Axial stiffness measurement A constant flow rate of 100-1300 μL/minute, generated by a syringe pump (NE-1002X, Pump System Inc.) and resulting in approximately 5-65 pN as the particle drag force, was utilized to create hydrodynamic forces to stretch the DNA nanobeams. The elbow Luer connector male (Ibidi, 10802) and tube adapter set (Ibidi, 10831) were used to connect flow chambers to syringe pump. The DNA beams attached to the particles were imaged at 0.2 second interval for 3.5 minutes. The particle coordinates were tracked to measure displacement using TrackMate (FIJI-ImageJ, NIH), and subsequently used to evaluate drag forces at the interface experienced by the particles. The drag force at the interface of the particle was calculated by the equation assuming the interfacial particle at the fluid-fluid interface (Park, S., et al., (2019) Sci. Rep., 101038/s41598-019-49592-1, incorporated. herein by reference in its entirety). The slope of the applied force over the displacement was determined by linear fitting to obtain the value of EA.

### Example 1

### Design of DNA Nanobeams with Tailored Densities of Nicks-HJs

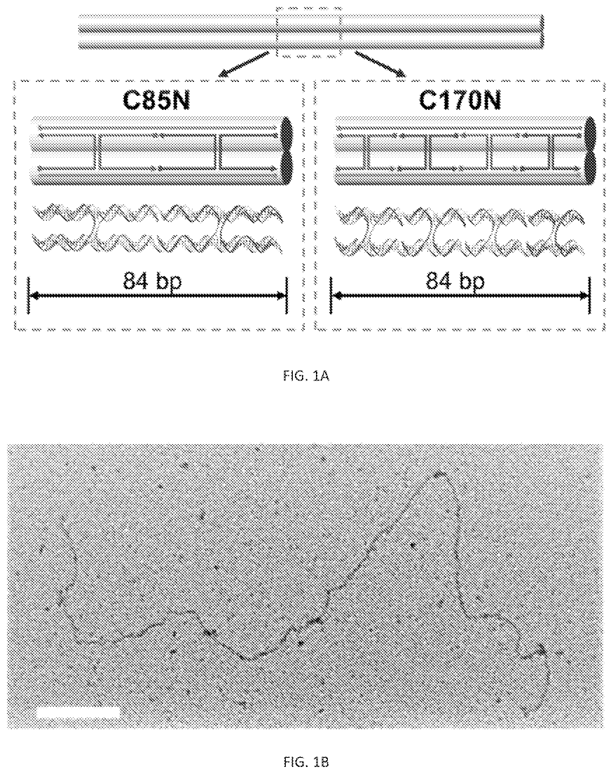

To evaluate the effect of nicks and HJs on the axial stiffness of DNA nanostructures, two designs of DNA origami nanobeams were synthesized and then modified to produce four different constructs. All nanobeams consisted of two DNA duplexes connected through HJs, creating a series of periodic DX-tiles. The first design, C170N, had a double-crossover every 21 nucleotides (170 HJs total), while in the second design, C85N, the distance between successive His was set to be 42 nucleotides (85 HJs total). The crossover spacing was chosen to ensure that the beams have negligible pre-strains caused by under- or over-twisting. Both these HJs densities created a domain gap between neighboring helices, mainly caused by electrostatic interactions, but this effect was eliminated under large stretching forces. In both designs, shown in FIG. 1A, the two helices were nicked in each center between consecutive crossovers, resulting in their total number of nicks being 340 and 170 respectively. Using T4 DNA ligase in both constructs led to two more nanobeams, namely constructs C170L and C85L, which had all nicks ligated and the same crossover densities as the C170N and C85N correspondingly. This allowed evaluation of the uncoupled effects of HJs and nicks by measuring the stretching stiffness of each DNA nanobeam. All constructs were synthesized using a one-pot annealing process overnight (12, 13). The simplicity of the designs facilitated a synthesis process with very high yield, which for single-layer DNA origami is typically close to 100%. The successful assembly was confirmed by gel-electrophoresis while transmission electron microscopy (TEM) imaging (FIG. 1B) verified that the length of the synthesized nanobeams was approximately 1.2 μm, corresponding to half the length of the single-stranded M13mp18 scaffold.

### Example 2

### Stretching Experiments and Axial Stiffness Measurements

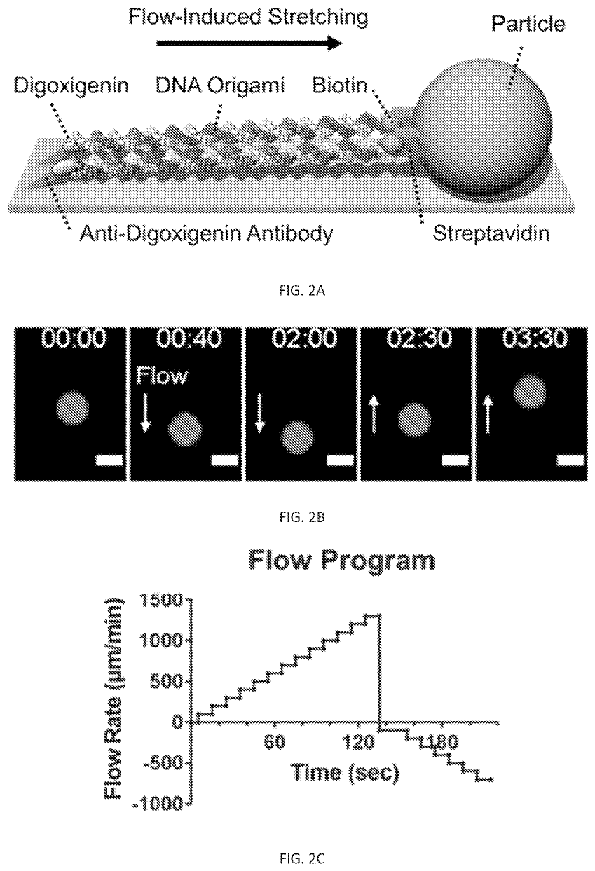

The axial stiffness measurement of the DNA nanobeams was performed using a microfluidic platform (FIG. 2B), which is an established method to probe the mechanical properties of DNA molecules (Wang, J. and Lu, C. (2007) J. Appl. Phys., 102, 074703, Hirano, K., et al., (2018) J. Chem. Phys., 149, 165101, and Onoshima, D. and Baba, Y. (2017) In. Humana Press, New York, NY, pp. 105-111, each incorporated herein by reference in their entirety) as an alternative to optical tweezers. Prior to the measurement, the DNA nanobeams were first immobilized on an anti-digoxigenin antibody-grafted glass surface by one of its extremities (two unhybridized “linker” sequences) conjugated with digoxigenin molecules. The stretching force was applied hydrodynamically through a micron-sized particle bound to the free end of the DNA nanobeam (FIG. 2B), while the buffer was passed through the microfluidic device. The binding between the particle and the DNA. nanobeam was facilitated by the streptavidin-biotin interaction. By adjusting the flow rate (FIG. 2C), the stretching force magnitude exerted on the DNA origami can be modulated ranging from 1 Pico Newton (pN) to 50 pN. The stretching force was applied stepwise, with 5 pN as the starting magnitude and a 5 pN unit increment every 10 seconds, until reaching a magnitude of 65 pN. The displacement of the micron-sized particle was tracked (FIG. 2D) and the elongation of the DNA origami as a function of the applied force was recorded. The force-displacement curves F-δ for each DNA origami construct was plotted, the slope was extracted and the axial stiffness k was calculated (slope of the curve multiplied by the origami length). Since this focused only in the apparent axial stiffness of DNA origami past the entropic elasticity regime, the slopes for forces larger than 15 pN were measured. At these force levels, the DNA nanobeams were fully straightened and stretched beyond their original length.

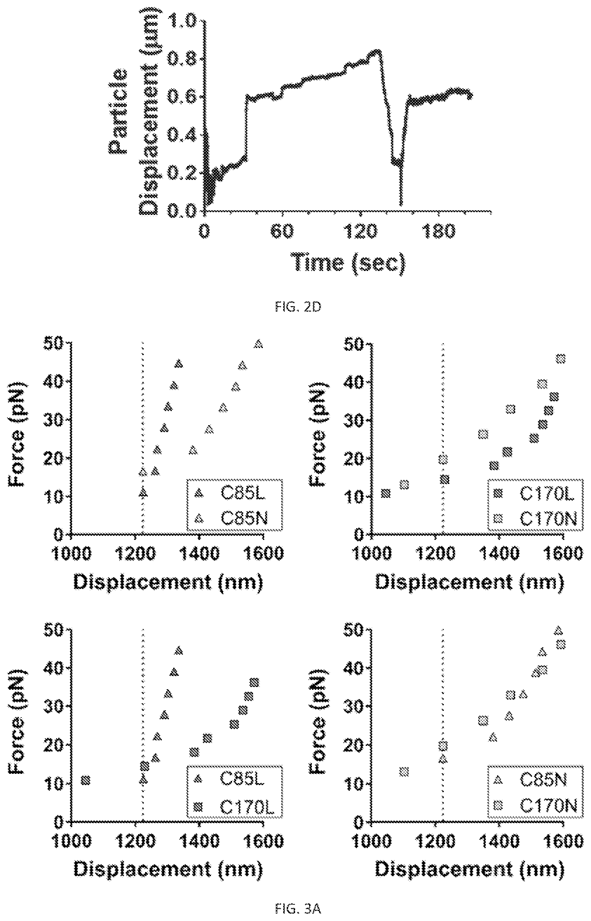

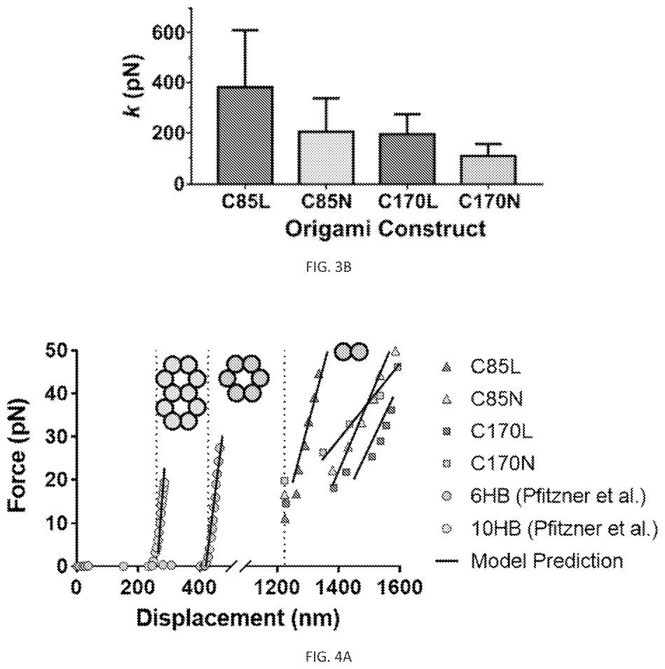

Typical responses from four measurements with values very close to the collective mean of each DNA construct are shown in FIG. 3A. The two plots on the top show the difference between the responses of ligated and nicked assemblies with the same number of HJs respectively (85 and 170). The two plots at the bottom showcase the effect of the HJs density on the response of origami with (left plot) and without (right plot) the presence of nicks. The collective results showed that the stiffest response corresponded to the ligated nanobeam with the smaller number of HJs (C851L) while the more compliant beam was the nicked origami with the highest density of HJs (C170N). The mean values and standard deviations of the axial stiffness measured from all the responses of the C85L, C85N, C170L, and C170N DNA origami beams are listed in Table 1 (see FIG. 3B). An 80% increase of axial stiffness was observed for the ligated nanobeams compared to the corresponding values of their counterparts with the nicked helices. This comparison illustrated that the increased number of nicks significantly reduces the stiffness of the origami beams. Furthermore, reducing the number of HJs by 50% resulted in a near two-fold increase of the origami stiffness. This counter-intuitive effect indicated that adding HJs in DNA nanostructures resulted in an expected significant increase in their bending rigidity, it comes with a substantial decrease of their stretching stiffness. This loss of stiffness was examined further using continuum modeling and finite element simulations corresponding to the same designs tested experimentally.

TABLE 1
Measurement and Model Prediction Values of Axial Stiffness
for DNA nanobeams with different HJs and nicks

| | | | | | | Mean | Model |
|---|---|---|---|---|---|---|---|
| | Length | #HJs | | # Nicks | | Stiffness ± | Result |
| Construct | (nm) | (#/21 bp) | | (#/21 bp) | | SD (pN) | (pN) |
| C85L | 1224 | 85 | (0.5) | 0 | (0) | 382 ± 227 | 350 |
| C85N | 1224 | 85 | (0.5) | 170 | (0.5) | 206 ± 131 | 205 |
| C170L | 1224 | 170 | (1) | 0 | (0) | 181 ± 82 | 181 |
| C170N | 1224 | 170 | (1) | 340 | (1) | 108 ± 47 | 108 |
| 6HB* | 428 | 336 | (2) | 168 | (0.5) | 337 | 274 |
| 10HB* | 257 | 360 | (2) | 180 | (0.5) | 341 | 409 |

*See Pfitzner, E., Wachauf, C., Kilchherr, F., Pelz, B., Shih, W. M., Rief, M. and Dietz, H. (2013). Angew. Chemie, 125, 7920-7925, incorporated herein by reference in its entirety.

### Example 3

### Modeling and Virtual Stretching of DNA Origami

To further examine and elucidate the experimental measurements, computational models that treat each helix as a continuum elastic rod with the effective geometric and material properties of B-form DNA were used. The sequence-based representations were parsed into finite element models using a CanDo source code that translated each base-pair into a two-node beam element with elastic properties (stretching, bending and torsional stiffness) corresponding to B-form DNA (Castro, C. E., et al. (2011) Methods, 8, 221-229, Kim, D.-N., et al., (2012) Nucleic Acids Res., 40, 2862-2868, and Pan, K., et al., (2014) Nat. Commun., 5, 5578, each incorporated herein by reference in their entirety). All crossovers were assumed to rigidly constrain the neighboring helices. The beam elements corresponding to the locations of the HJs and the nicks were assigned axial stiffness values equal to αk and βk respectively, where k is the stretching stiffness of the (intact) double helix and α, β are constants to be determined. In essence, the two constants represented an effective local degradation of the axial stiffness at the locations of crossovers and nicks. The finite element software ABAQUS (Simulia) was used to apply external axial forces on each origami structure and numerically generate their force-extension curves. The nanostructural designs chosen here, i.e, two-helix beams with crossovers every 21/42 nucleotides, led to a minimal effect from bending and twisting of the DNA helices during stretching, which in other DNA assemblies can be significant.

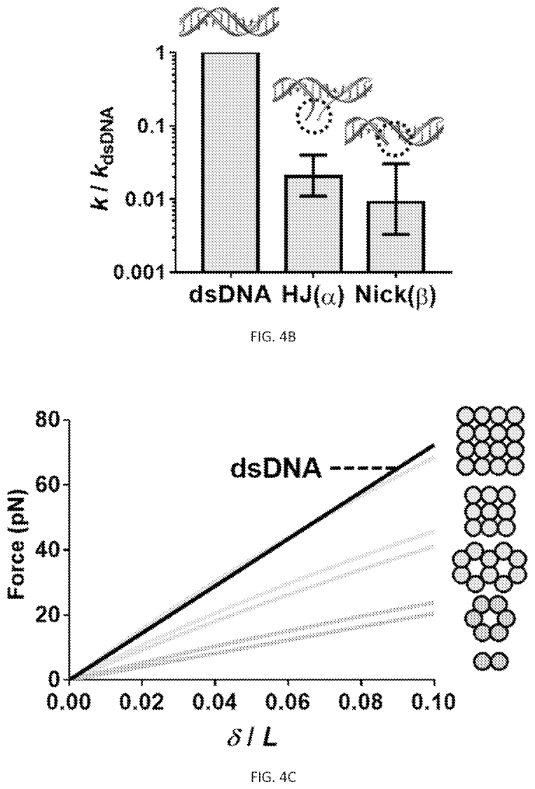

The local stiffness parameter β was first calibrated by an optimal fit between the numerical response and the average stiffness of the C170L nanobeam which does not have any nicks. The second parameter, α, was then evaluated by an optimal fit using the experimental data from the C170N construct. Subsequently, using the calibrated parameters, the force-extension curves were simulated for the remaining two nanobeams (C85L and C85N). The results of the numerical simulations, shown in FIG. 4A and also reported in Table 1, agreed extremely well with the corresponding experimental data. To further examine the validity of the numerical framework and its applicability to different origami designs, the stretching of a 6-helix DNA origami with an approximate length of 428 nm for which experimental results under tension were reported previously (Pfitzner, E., et al., (2013) Angew. Chemie, 125, 7920-7925, incorporated herein by reference in its entirety) was also simulated. The predicted response from the simulation, also shown in FIG. 4A and reported in Table 1, agreed with the measurements made previously.

The relative axial stiffness where the HJs and the nicks occur, represented by the fitted values of α and β, are 0.023 and 0.0081, respectively (FIG. 4B), in comparison to the dsDNA stiffness. The modeling results suggested that the local stiffness for both HJs and nicks was two orders of magnitude smaller than one corresponding to the intact double helix. In order to fully understand the origins of this stiffness reduction a systematic study that accounts for all nanoscopic interactions would be necessary but herein it was attributed mainly to the differences of the mechanical behavior between ssDNA and dsDNA. At the force regime examined here (10-25 pN/helix), the duplexes were in the enthalpic elasticity region and thus fairly stiff (see Smith, S. B., Cui, Y. and Bustamante, C. (1996) Science, 271, 795-799, incorporated herein by reference in its entirety). However, at the same force levels, the short segments of ssDNA in nicks and at HJs, were still in the entropic regime with an extremely low apparent stiffness, indicating that the tensile loads would be able to eliminate any stacking interactions between bases, thus making the nicked helix to behave locally as ssDNA under low entropic forces.

The same modeling framework was next used to understand how the local loss of axial stiffness from HJs and nicks correlates to the macroscopic stiffness of assemblies with different number of neighboring helices. Tensile simulations were performed on origami beams with cross-sections consisting of 2-16 double helices packed on both honeycomb and square lattices. The force-displacement curves are shown in FIG. 4C and the most striking result was that almost all origami were less stiff than the intact (e.g., having no nicks) double helix. It was also interesting, and counter-intuitive, that the relation between the apparent stiffness k of each rod and the number N of the helices was nonlinear. This can be seen in FIG. 4D where, for example, the 6-helix rod appeared to be slightly stiffer than the 2-helix one, but much less stiff than the 9- and 10-helix bundles. The same figure also shows that in order to reach values of axial stiffness attained by dsDNA, an assembly of at least 16 helices was required. Strikingly, despite the fact that adding helices in DNA origami increases their overall bending rigidity and the corresponding persistence length, it decreased their apparent axial stiffness.

### References:

1. Pinheiro, A. V., Han, D., Shih, W. M. and Yan, H. (2011) Challenges and opportunities for structural DNA nanotechnology. Nat. Nanotechnol., 6, 763-772.
2. Seeman, N. C. (1998) DNA nanotechnology: Novel DNA constructions. Annu. Rev. Biophys. Biomol. Struct., 27, 225-248.
3. Zhang, F., Nangreave, J., Liu, Y. and Yan, H. (2014) Structural DNA nanotechnology: State of the art and future perspective. J. Am. Chem. Soc., 136,11198-11211.
4. Seeman, N. C. and Sleiman, H. F (2017) DNA nanotechnology. Nat. Rev. Mater., 3, 17068.
5. Linko, V. and Dietz, H. (2013) The enabled state of DNA nanotechnology. Curr. Biotechnol., 24, 555-561.
6. Wamhoff, E.-C., Banal, J. L, Bricker, W. P., Shepherd, T. R., Parsons, M. F., Stone, M. B., Jun, H., Wang, X. and Bathe, M. (2019) Programming Structured DNA Assemblies to Probe Biophysical Processes. Annu. Rev. Biophys., 48, 395-419.
7. Jiang, Q., Song, C., Nangreave, J., Lin, L., Qiu, D., Zou, G., Yan, H., et at. (2012) DNA origami as a carrier for circumvention of drug resistance. J. Am. Chem. Soc., 134, 13396-13403.
8. Zhao, Y. X., Shaw, A., Zeng, X., Benson, E., Nyström, A. M. and Högberg, B. (2012) DNA origami delivery system for cancer therapy with tunable release properties. ACS Nano, 6, 8684-8691.
9. Ke, Y., Castro, C. and Choi, J. H. (2018) Structural DNA Nanotechnology: Artificial Nanostructures for Biomedical Research. Annu. Rev. Blamed. Eng., 20, 375-401.
10. Beater, S., Raab, M. and Tinnefeld, P. (2014) Toward quantitative fluorescence microscopy with DNA origami nanorulers. In Methods in Cell Biology. Academic Press Inc., Vol. 123, pp. 449-466.
11. Chao, J., Zhang, H., Xing, Y., Li, Q., Wang, L., Wang, L., and Fan, C. (2018) Programming DNA origami assembly for shape-resolved nanomechanical imaging labels. Nat. Protoc., 13, 1569-1585.
12. Rothermund, P. W. K. (2006) Folding DNA to create nanoscale shapes and patterns. Nature, 440, 297-302.
13. Castro, C. F., Kilchherr, F., Kim, D.-N., Shiao, E. L., Wauer, T., Wortmann, P., Bathe, M, and Dietz H. (2011) A primer to scaffolded DNA origami. Nat. Methods, 8, 221-229.
14. Hagerman, P. J. (1988) Flexibility of DNA. Annu. Rev. Biophys. Biophys. Chem., 17. 265-286.
15. Han, D., Pal, S., Nangreave, J., Deng, Z., Liu, Y. and Yan, H. (2011) DNA origami with complex curvatures in three-dimensional space. Science, 332, 342-346.
16. Hong, F., Zhang, F., Liu, Y. and Yan, H. (2017) DNA Origami: Scaffolds for Creating Higher Order Structures. Chem. Rev., 10.102/acs.chemrev.6b00825.
17. Wang, P., Meyer, T. A., Dutta, P. K. and Ke, Y. (2017) The Beauty and Utility of DNA Origami. Chem, 2, 359-382.
18. Sa-Ardyen, P., Vologodskii, A. V. and Seeman, N. C. (2003) The flexibility of DNA double crossover molecules. Biophys. J., 84, 3829-3837.
19. Veneziano, R., Ratanalert, S., Zhang, K., Zhang, F., Chiu, W. and Bathe, M. (2016) Designer nanoscale DNA assemblies programmed from the top down. Science, 352, 1534-1534.
20. Jun, H., Zhang, F., Shepherd, T., Ratanalert, S., Qi, X., Yan, H. and. Bathe, M. (2019) Autonomously designed free-form 2D DNA origami. Sci. Adv., 5, eaav0655.
21. Zhang, F., Li, W., Hunt, A., Liu, Y. and Yan, H. (2016) Self-Assembly of Complex DNA Tessellations by Using Low-Symmetry Multi-arm DNA Tiles. Angew. Chemie Int. Ed., 55, 8860-8863.
22. He, Y., Ye, T., Su, M., Zhang, C., Ribbe, A. E., Jiang, W. and Mao, C. (2008) Hierarchical self-assembly of DNA into symmetric supramolecular polyhedra. Nature, 452, 198-201.

23. Yoo, J. and Aksimentiev, A. (2013) In situ structure and dynamics of DNA origami determined through molecular dynamics simulations. Proc. Natl. Acad. Sci. U.S.A., 110, 20099-20104.
24. Castro, C. E., Marras, A. E., Zhou, L, and Johnson). (2015) Mechanical design of DNA nanostructures. Nanoscale, 7, 5913-5921.
25. Shrestha, P., Emura, T., Koirala, D. Cui, Y., Hidaka, K., Maximuck, W. J., Endo, M., Sugiyama, H. and Mao, H. (2016) Mechanical properties of DNA origami nanoassemblies are determined by Holiday junction mechanophores. Nucleic Acids Res., 44, 6574-6582.
26. Lee, J. Y., Lee, C., Lee, J. G., Yagyu, R, Tabata, O. and Kim, D.-N. (2019) Investigating the sequence-dependent mechanical properties of DNA nicks for applications in twisted DNA nanostructure design. Nucleic Acids Res., 47, 93-102.
27, Lee, C., Lee, J. Y. and Kim, D. N. (2017) Polymorphic design of DNA origami structures through mechanical control of modular components. Nat. Commun., 8, 1-8.
28. Marras, A. E., Zhou, L., Su, H. J. and Castro, C. E. (2015) Programmable motion of DNA origami mechanisms. Proc. Natl. Acad. Sci. U.S.A. 112. 713-718.
29. Zhou, L., Marras, A. E., Su, H. J, and Castro, C. E. (2015) Direct design of an energy landscape with bistable DNA origami mechanisms. Nano Lett., 15, 1815-1821.
30. Zhou, L., Marras, A. E., and Castro, C. E. (2014) DNA Origami Compliant Nanostructures with Tunable Mechanical Properties. ACS Nano, 8, 27-34.
31. Lee, C., Kim, K. S., Kim, Y. J., Lee, J. Y. and. Kim, D. N, (2019) Tailoring the Mechanical Stiffness of DNA Nanostructures Using Engineered Defects. ACS Nano, 13, 8329-8336.
32. Dietz, H., Douglas, S. M. and Shih, W. M. (2009) Folding DNA into twisted and curved nanoscale shapes. Science, 325, 725-30.
33. Kauert, D. J., Kurth, T., Siedel, T, and Seidel, R. (2011) Direct Mechanical Measurements Reveal the Material Properties of Three-Dimensional DNA Origami. Nano Lett., 11, 5558-5563.
34. Schiffels, D., Liedl, T. and Fygenson, D. K. (2013) Nanoscale structure and microscale stiffness of DNA nanotuhes. ACS Nano, 7, 6700-6710.
35. Plesa, C., Ananth, A. N., Linko, V., Katan, A. J., Dietz, H. and Dekker, C. (2014) Ionic permeability and mechanical properties of DNA origami nanoplates on solid-state nanopores. ACS Nano, 8, 33-43.
36. Sun, W., Boulais, E., Hakobyan, Y., Guan, A., Bathe, M. and Yin, P. (2014) Casting inorganic structures with DNA molds. Science, 346.
37. Wang, P., Gaitanaros, S., Lee, S., Bathe, M., Shih, W. M. and Ke, Y, (2016) Programming Self-Assembly of DNA Origami Honeycomb Two-Dimensional Lattices and Plasmonic Metamaterials. J. Am. Chem. Soc., 138, 7733-7740.
38. Pfitzner, E., Wachauf, C., Kilchherr, F., Pelz, B., Shih, W. M., Rief, M. and Dietz, H. (2013) Rigid DNA Beams for High-Resolution Single-Molecule Mechanics. Angew. Chemie, 125, 7920-7925.
39. Wang, J. and Lu, C. (2007) Single molecule λ-DNA stretching studied by microfluidics and single particle tracking. J. Appl. Phys., 102, 074703.
40. Tinevez, J. Y., Perry, N., Schindelin, J., Hoopes, G. M., Reynolds, G. D., Laplantine, E., Bednarek, S. Y., Shorte, S. L. and. Elicein, K. W, (2017) TrackMate: An open and extensible platform for single-particle tracking. Methods, 115, 80-90.
41. Park, S., Joo, Y. K. and Chen, Y. (2019) Versatile and High-throughput Force Measurement Platform for Dorsal Cell Mechanics. Sci. Rep., 10.1038/s41598-019-49592-1.
42. Hirano, K., lwaki, T., Ishido, T., Yoshikawa, Y., Naruse, K. and Yoshikawa, K. (2018) Stretching of single DNA molecules caused by accelerating flow on a microchip. J. Chem. Phys., 149, 165101.
43. Onoshima, D. and Baba, Y. (2017) Microfluidic DNA Stretching Device for Single-Molecule Diagnostics, In. Humana Press, New York, NY, pp. 105-111.
44. Silver, J., Li, Z. and Neuman, K. (2015) Tethered-bead, immune sandwich assay. Biosens, Bioelectron., 63, 117-123.
45. Kim, D.-N., Kilchherr, F., Dietz, H. and Bathe, M. (2012) Quantitative prediction of 3D solution shape and flexibility of nucleic acid nanostructures. Nucleic Acids Res., 40, 2862-2868.
46. Pan, K., Kim, D.-N., Zhang, F., Adendorff, M. R., Yan, H. and Bathe, M. (2014) Lattice-free prediction of three-dimensional structure of programmed DNA assemblies, Nat. Common., 5,5578.
47. Smith, S. B., Cui, Y. and Bustamante, C. (1996) Overstretching B-DNA: The elastic response of individual double-stranded and single-stranded DNA molecules. Science, 27L 795-799.
48. Dutta, P. K., Zhang, Y., Blanchard, A. T., Ge, C., Rushdi, M., Weiss, K., Zhu, C., Ke, Y. and Salaita, K. (2018) Programmable Multivalent DNA-Origami Tension Probes for Reporting Cellular Traction Forces. Nano Lett., 18, 4803-4811.
49. Blanchard, A. T. and Salaita, K. (2019) Emerging uses of DNA mechanical devices. Science, 365, 1080-1081.
50. Glazier, R., Brockman, J. M., Bartle, E., Mattheyses, A. L., Destaing, O. and Salaita, K. (2019) DNA mechanotechnology reveals that integrin receptors apply pN forces in podosomes on fluid substrates. Nat. Common., 10, 1-13.
51. Ma, V. P. and Salaita, K. (2019) DNA Nanotechnology as an Emerging Tool to Study. Mechanotransduction in Living Systems. Small, 15, 1900961.
52. Ma, R., Kellner, A. V., Ma., V. P. Y., Deal, B, R., Brockman, J. M. and Salaita, K. (2019) DNA probes that store mechanical information reveal transient piconewton forces applied by T cells. Proc. Natl. Acad. Sci. U.S.A., 116, 16949-16954.
53. Liu, Y. & Zen, G. Cancer and innate immune system interactions: Translational potentials for cancer immunotherapy. J. Immunother. 35, 299-308 (2012).
54. Adams, T. & Kortylewski, M. The revival of CpG oligonucleotide-based cancer immunotherapies. Contemp. Oncol. 2018, 56-60 (2018).
55. Pillarisetty, V. G., Katz, S. C., Bleier, J. I., Shah, A. B., Dematteo, R. P. & Goldman, M. Natural killer dendritic cells have both antigen presenting and lytic function and in response to CpG produce IFN-gamma via autocrine IL-12. J. Immunol. 174, 2612-8 (2005).
56. Krieg, A. M. CpG Still Rocks! Update on an Accidental Drug, Nucleic Acid Ther. 22, 77-89 (2012).
57. Krieg, A. M. Therapeutic potential of Toll-like receptor 9 activation. Nat. Rev. Drug Discov. 5, 471-484 (2006).
58. Krogmann, A. O., Lüsebrink, E., Steinmetz, M., Asdonk, T., Lahrmann, C., Lütjohann, D., Nickenig, G. & Zimmer, S. Proinflammatory Stimulation of Toll-Like Receptor 9 with High Dose CpG ODN 1826 Impairs Endothelial Regeneration and Promotes Atherosclerosis in Mice. PLoS One 11, e0146326 (2016).
59. Indra, I., Undyala, V., Kandow, C., Thirumurthi, U., Dembo, M. & Beningo, K. A. An in vitro correlation of mechanical forces and metastatic capacity. Phys. Biol. 8, 015015 (2011).
60. Levental, K. R., Yu, H., Kass, L., Lakins, J. N., Egeblad, M., Erler, J. T., Fong, S. F. T., Csiszar, K., Giaccia, A., Weninger, W., Yamauchi, M., Gasser, D. L. & Weaver, V. M. Matrix Crosslinking Forces Tumor Progression by Enhancing Integrin Signaling. Cell 139, 891-906 (2009).
61, Goldstein, M. J., Varghese, B., Brody, J. D., Rajapaksa, R., Kohrt, H., Czerwinski, D. K., Levy, S. & Levy, R. A CpG-loaded tumor cell vaccine induces antitumor CD4+ T cells that are

It is understood that the foregoing detailed description and accompanying examples are merely illustrative and are not to be taken as limitations upon the scope of the invention, which is defined solely by the appended claims and their equivalents.

Various changes and modifications to the disclosed embodiments will be apparent to those skilled in the art. Such changes and modifications, including without limitation those relating to the chemical structures, substituents, derivatives, intermediates, syntheses, compositions, formulations, or methods of use of the invention, may be made without departing from the spirit and scope thereof.

What is claimed is:

1. A device comprising a plurality of DNA nanostructures each tethered at a distal end to an implantable substrate, wherein each of the DNA nanostructures comprises:
a cell ligand at a proximal end; and
CpG oligodeoxynucleotides (CpG ODNs) configured to be released when the DNA nanostructure is under force.

2. The device of claim 1, wherein the plurality of DNA nanostructures comprises DNA nanobeams.

3. The device of claim 1 or claim 2, wherein the CpG ODNs are staple strands within an individual DNA nanostructure.

4. The device of any of claims 1-3, wherein the implantable substrate comprises a catheter-like device, a shunt, or a porous silicone gel.

5. The device of any of claims 1-3, wherein the implantable substrate is a microparticle.

6. The device of claim 5, wherein the microparticle comprises a targeting compound, wherein the targeting compound is configured to interact with a target cell type or tissue.

7. The device of any of claims 1-6. wherein the plurality of DNA nanostructures is tethered to the implantable substrate with a covalent bond.

8. The device of any of claims 1-6, wherein the plurality of DNA nanostructures is tethered to the implantable substrate with a noncovalent bond.

9. The device of any of claims 1-8, wherein each of the plurality of DNA nanostructures comprises on the distal end a moiety configured to interact with an agent on an outer surface of implantable substrate.

10. The device of claim 9, wherein the moiety and the agent are a binding pair.

11. The device of claim 9, wherein the moiety and the agent are reactive functional groups.

12. The device any of claims 1-11, wherein the cell ligand is a molecule capable of binding integrin or cytotoxic T-lymphocyte-associated protein 4 (CTLA-4).

13. The device of any of claims 1-12, wherein the cell ligand comprises fibronectin, an arginine-glycine-aspartate (RCD) peptide, or cluster of differentiation 80 protein (CD80).

14. The device of any of claims 1-13, wherein the device is implantable or insertable into a subject.

15. A method of treating cancer in a subject comprising implanting the device of any of claims 1-14 in a subject in need thereof.

16. The method of claim 15, wherein the device is implanted intratumorally.

17. The method of claim 15 or 16, wherein the device is implanted post-tumor resection.

18. The method of any of claims 15-17, further comprising administration of an additional therapy selected from the group consisting of immunotherapy, radiotherapy, administration of a second chemotherapeutic agent, or a combination thereof.

19. The method of any of claims 15-18, wherein the cancer is a solid tumor.

20. A system comprising:
an implantable substrate;
a plurality of DNA nanostructures comprising CpG oligodeoxynucleotides CpG ODNs) configured to be released when the DNA nanostructure is under force; and
a cell ligand,
wherein each of the DNA nanostructures is configured to be tethered to the implantable substrate at a distal end and the cell ligand at a proximal end.

21. The system of claim 20, wherein the plurality of DNA nanostructures comprises DNA nanobeams.

22. The system of claim 20 or 21, wherein the CpG ODNs are staple strands within an individual DNA nanostructure.

23. The system of any of claims 20-22, wherein the implantable substrate comprises a catheter-like device, a shunt, or a porous silicone gel.

24. The device of any of claims 20-22, wherein the implantable substrate is a microparticle.

25. The device of claim 24, wherein the microparticle comprises a targeting compound, wherein the targeting compound is configured to interact with a target cell type or tissue.

26. The system of any of claims 20-25, wherein each of the plurality of DNA nanostructures comprises on the distal end a moiety configured to interact with an agent on an outer surface of implantable substrate.

27. The system of claim 26, wherein the moiety and the agent are a binding pair.

28. The system of claim 27, wherein the moiety and the agent are reactive functional groups.

29. The system of any of claims 20-28, wherein the cell ligand is a molecule capable of binding integrin or cytotoxic T-lymphocyte-associated protein 4 (CTLA-4).

30. The system of any of claims 20-29, wherein the cell ligand comprises fibronectin, an arginine-glycine-aspartate (RGD) peptide, or cluster of differentiation 80 protein (CD80).
